(19) United States
(12) Patent Application Publication
(10) Pub. No.: US 2004/0070993 A1
(43) Pub. Date: Apr. 15, 2004

(54) Melioration of installation structure for illuminating pen

(75) Inventor: Hsia Ling Wang, Taipei (TW)

Correspondence Address: BACON & THOMAS, PLLC, 625 SLATERS LANE, FOURTH FLOOR, ALEXANDRIA, VA, 22314

(73) Assignee: Viceory Seart Industrial Co., Ltd., Taipei (TW)

(21) Appl. No.: 10/268,794

(22) Filed: Oct. 11, 2002

(51) Int. Cl. A61B 1/24

(52) U.S. Cl. 362/577

(57) ABSTRACT

The present invention related to an installation structure for illuminating pen, which is an melioration to the conventional pens. A press section is fixed on top of the pen which controlling the extrusion and retraction of the refill. A flexible handling section is designed at the bottom section of the pen, such easy-gripped design gives compatible handling moderation. The characteristic of present invention is where the pen is fixes with a build-in illuminant, the fixture will then liberates the illuminant continually by pressing the press section hence such invention does not only fit the stationary purpose design, but also bring in new-day leading fashion ideas.

### BACKGROUND OF THE INVENTION

The present invention related to an installing structure of a pen, in particular, such meliorated pen fits in the purpose stationary design and contains a fashionable illuminant idea design.

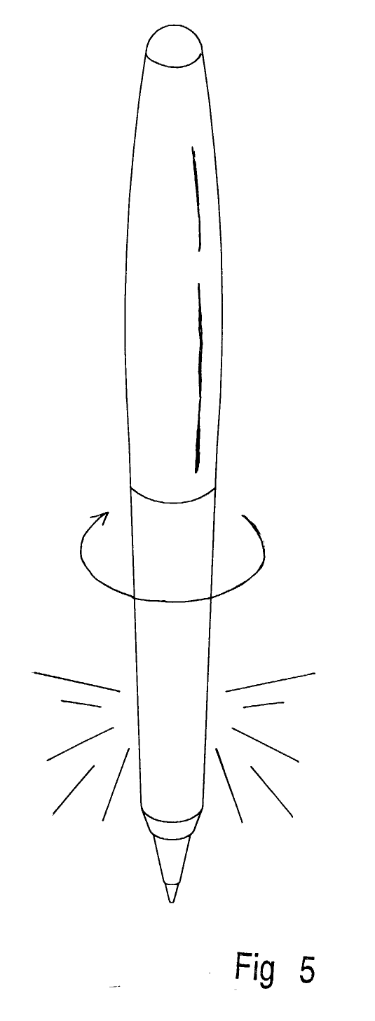

A conventional pen usually made with a holder and refill in a wide and variety design choices. The conventional pen design mainly based on it's utility, and its decorative appearance are unusually combined, whereas the current illuminating pen is designed with a build-in illuminant unit, by shifting the body unit enable the extrusion and retraction of the refill.. When the body unit is shift one side, the refill will be stretched out enable the writing purpose; whereas the body unit is shift to the other side, the refill will be shrink within enable the protection of refill. The illuminant will be liberated as the refill stretched out offers the lighting effect at the same time, as refer to FIG. 5.

Furthermore, such structure design is utilized and has been accepted by the general public for it's fresh and fashionable ideas, however, any utility design is possibly replaceable constantly due to the competitive market environment, a simple change on the out looks and visual effect will not be able to fit the general public's preference. Thus, people are thinking constantly of all the possible new and utility purpose design products to be the market leader.

Accordingly, for the above reason, the present invention offers a melioration of installation structure for illuminating pen in order to fit the general public's satisfaction, such invention eliminated the defect of simple and single design in the illuminating pen on current market. The present invention related to an installation structure for illuminating pen enhance a variety choice of vision design in pens for the general public. Thus, the present invention related to a melioration of installation structure for illuminating pen, a manual press control design fit in the writing purpose and fashionable visual effect.

The present invention can be best understood through the following description and accompanying drawings wherein:

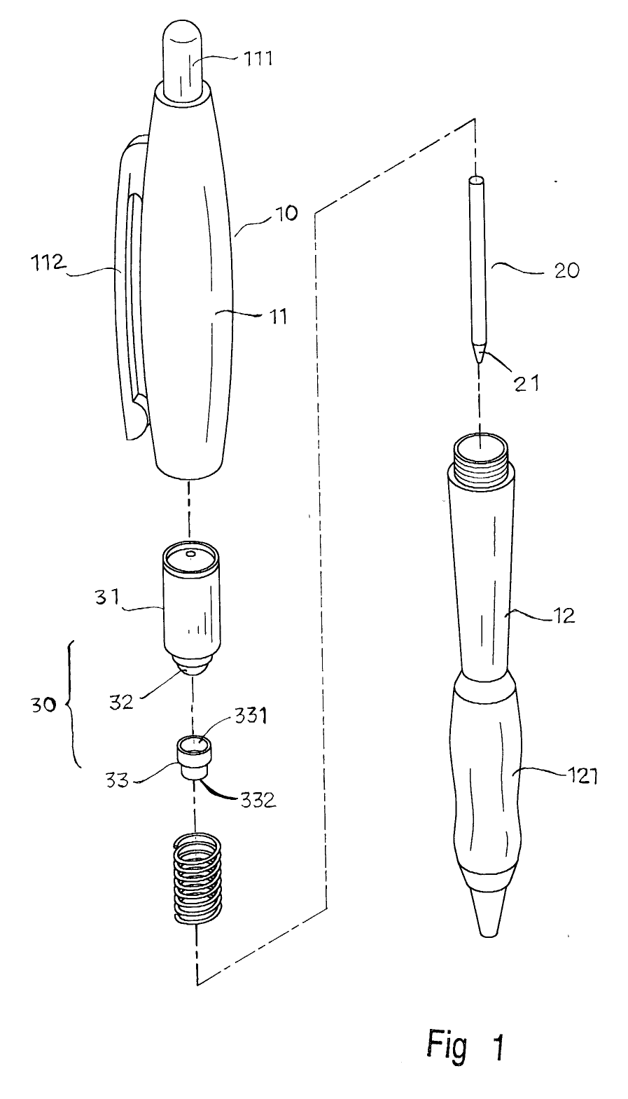

FIG. 1 shows a disassembled view of present invention

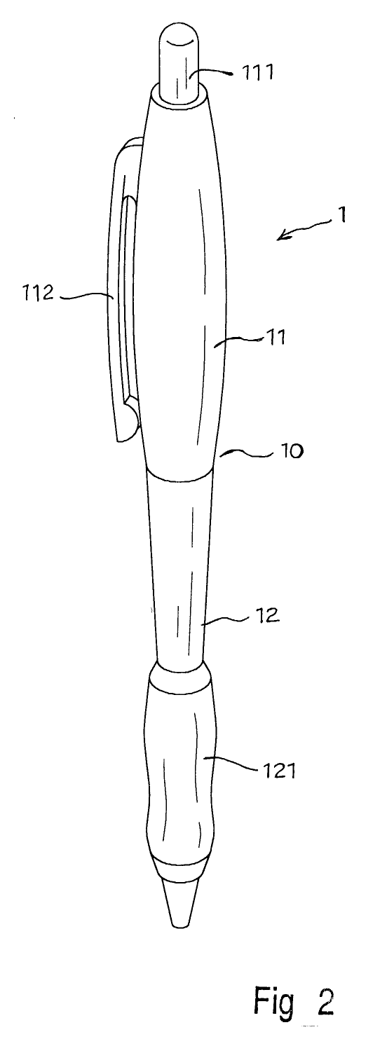

FIG. 2 shows an assembled view of present invention

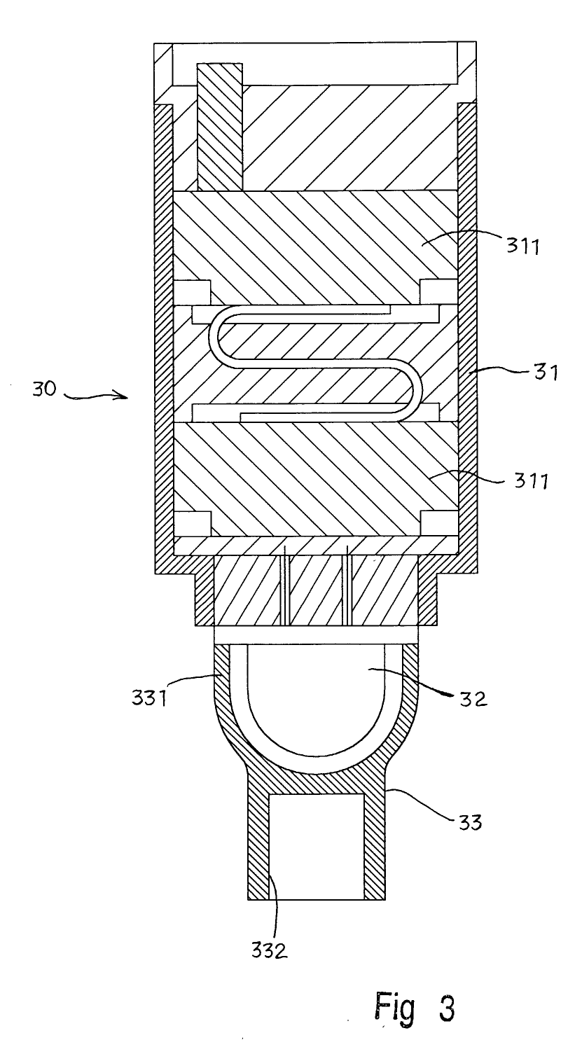

FIG. 3 shows a partial exploded view of present invention

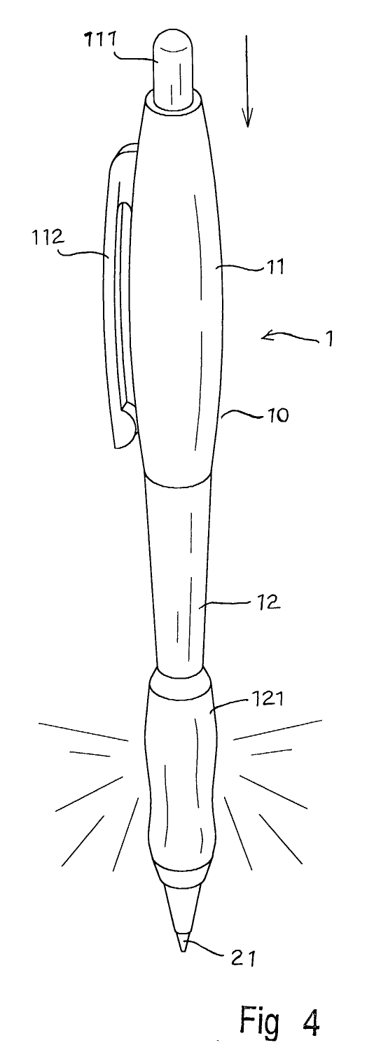

FIG. 4 shows the application of present invention

FIG. 5 shows the application of conventional illuminating pen

### DETAILED DESCRIPTION OF THE PREFERRED EMBODIMENTS

The present invention related to an installing structure of a pen, in particular, such meliorated pen fits in the purpose stationary design and contains a fashionable illuminant idea design. The present invention includes a holder (10), refill (20) and illuminant (30).

The holder (10) is constructed by the top and bottom sleeve (11, 12) where screw them together, the top sleeve (11) contains a press section (111) on the top end and a clip (112) on the side enable the to stabilized the pen unit (1) as user preferred. The bottom sleeve (12) is designed with a soft easy-gripped handling section (121) in order for user to gain extra comfort when using it. The refill (20) is fixed within the holder (10), where one end build with a pen-point (21) in sharp shape, and connection to illuminant (30) in the other end. The illuminant includes a socket (31) with a mercury battery (311), the bottom end connected to a second illuminant (32) followed by a connecting unit (33). The connecting unit (33) is fixed with a U shape cap (331) which allows the embedment with the second illuminant (32). The cap (332) is attached to another cap (331) allow the connection to the refill (20).

Accordingly, on the application, by press down the press section (111) on the top sleeve (11) of holder (10) manually, allows the extrusion and retraction of the refill (20). As press down the press section (111), the illuminant will be activated hence the second illuminant (32) will liberates the lights at the same time, the illuminant will be deactivated by repeating the same instruction again. The pen clip (112) allows to stabilize such compact pen unit (1) inside the pocket or where is appropriate enhance the efficiency and effectiveness of such pen.

The single manual control of present invention allow the controlling of extrusion and retraction of the refill (20) and active the illuminant at the same time hence to replace the convention way of controlling by shifting round the body pen. Such characteristic design does not only offer a utility design, but also a new and fashionable leading idea.

What is claimed is:

1. A melioration of installation structure for illuminating pen includes the holder and a refill; the pen holder is formed by a top and bottom sleeve. The press section is build on top of the top sleeve where allowed the extrusion and retraction of the refill, and the bottom sleeve (12) is designed with a soft easy-gripped handling section (121) in order for user to gain extra comfort when using it. A illuminant is build inside the top sleeve, which can be activated by simply pressing the top section of the top sleeve such characteristic design does not only offer a utility design, but also a new and fashionable leading idea.

2. A melioration of installation structure for illuminating pen as claimed in claim 1, wherein the illuminant includes a socket with a mercury battery, the bottom end connected to a second illuminant followed by a connecting unit. The connecting unit is fixed with a U shaped cap which allows the embedment with the second illuminant. The cap is attached to another cap allow the connection to the refill.
